(19) United States
(12) Patent Application Publication
(10) Pub. No.: US 2020/0398112 A1
(43) Pub. Date: Dec. 24, 2020

(54) ATTENTION AND EXERTION BASED SAFETY MECHANISMS IN EXERCISE DEVICES

(71) Applicant: International Business Machines Corporation, Armonk, NY (US)

(72) Inventors: FANG LU, BILLERICA, MA (US); PAUL R BASTIDE, ASHLAND, MA (US); ISHWARYA RAJENDRABABU, HOBOKEN, NJ (US); SATHYANARAYANAN SRINIVASAN, CAMBRIDGE, MA (US)

(21) Appl. No.: 16/444,248

(22) Filed: Jun. 18, 2019

(51) Int. Cl. A63B 24/00; A63B 22/02; A63B 71/00; A61B 5/00; A61B 5/0205

(52) U.S. Cl. CPC A63B 24/0062; A63B 22/02; A63B 71/0054; A61B 5/024; A61B 5/02055; A63B 2024/0068; A63B 2024/0093; A61B 5/7275

(57) ABSTRACT

The present disclosure provides for attention and exertion based safety mechanisms in exercise devices by: identifying a user exercising on an exercise device while the exercise device is in a first state; monitoring a physical state and an attentiveness level of the user while the exercise device is in the first state, wherein monitoring the attentiveness level includes monitoring targets of a gaze of the user within a time window and a consistency of the gaze within the time window; determining, based on the physical state and the attentiveness level of the user, that the user is experiencing a risk of injury above a predefined threshold; and adjusting the exercise device from the first state to a second state to alleviate the risk of injury.

### BACKGROUND

The present invention relates to exercise devices, and more specifically, to monitoring a user of the exercise device to improve the user's safe use of that exercise device. When a user is exercising using a device such as a treadmill, elliptical machine, rowing machine, weight-lifting machine, etc., the settings of the device (e.g., speed, resistance, weight) or the duration of exercise (e.g., usage for m minutes, x repetitions) may exceed the capabilities of the user, and result in the user becoming injured. Additionally, a user who is distracted during an exercise may exhibit poor form, which can also lead to injury.

### SUMMARY

According to one embodiment of the present invention, attention and exertion based safety for exercise devices is provided via a method, comprising: identifying a user exercising on an exercise device while the exercise device is in a first state; monitoring a physical state and an attentiveness level of the user while the exercise device is in the first state, wherein monitoring the attentiveness level includes monitoring targets of a gaze of the user within a time window and a consistency of the gaze within the time window; determining, based on the physical state and the attentiveness level of the user, that the user is experiencing a risk of injury above a predefined threshold; and adjusting the exercise device from the first state to a second state to alleviate the risk of injury.

According to one embodiment of the present invention, attention and exertion based safety for exercise devices is provided via a system, comprising: a processor; and a memory including computer-readable program code that when executed by the processor causes the system to perform an operation comprising: identifying a user exercising on an exercise device while the exercise device is in a first state; monitoring a physical state and an attentiveness level of the user while the exercise device is in the first state, wherein monitoring the attentiveness level includes monitoring targets of a gaze of the user within a time window and a consistency of the gaze within the time window; determining, based on the physical state and the attentiveness level of the user, that the user is experiencing a risk of injury above a predefined threshold; and adjusting the exercise device from the first state to a second state to alleviate the risk of injury.

According to one embodiment of the present invention, attention and exertion based safety for exercise devices is provided via a computer program product comprising a computer-readable storage medium having computer-readable program code embodied therewith, the computer-readable program code executable by one or more computer processors to: identify a user exercising on an exercise device while the exercise device is in a first state; monitor a physical state and an attentiveness level of the user while the exercise device is in the first state, wherein monitoring the attentiveness level includes monitoring targets of a gaze of the user within a time window and a consistency of the gaze within the time window; determine, based on the physical state and the attentiveness level of the user, that the user is experiencing a risk of injury above a predefined threshold; and adjust the exercise device from the first state to a second state to alleviate the risk of injury.

### BRIEF DESCRIPTION OF THE SEVERAL VIEWS OF THE DRAWINGS

FIG. 1 illustrates two users on two exercise devices, according to embodiments of the present disclosure.

FIG. 2 illustrates a time lapse view of the range of motion for the first user and the second user, according to embodiments of the present disclosure.

FIG. 3 is a flowchart of a method for applying attention and exertion based safety mechanisms in exercise devices, according to embodiments of the present disclosure.

FIG. 4 illustrates a computing system, according to embodiments of the present disclosure.

### DETAILED DESCRIPTION

The present disclosure provides systems and methods to improve the safety of exercise equipment by monitoring the physical state and attentiveness level of the user to automatically adjust operations of the exercise equipment in accordance with the user's capabilities. By monitoring the usage of the exercise equipment in conjunction with the attentiveness level and physical state of the user, safer exercise equipment may be provided.

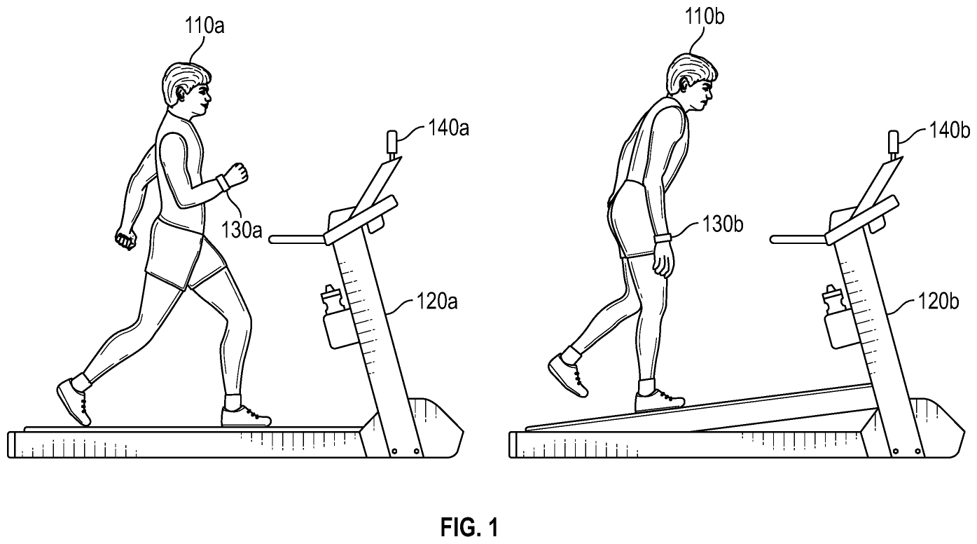

Consider, for example, a first user 110a and second user 110b (generally, users 110) of a respective first exercise device (ED) 120a and second ED 120b (generally, ED 120), illustrated in FIG. 1 as runners on treadmills. Each of the users 110 may wear an (optional) biometric device 130 (individually, first biometric device 130a and second biometric device 130b), and is monitored by a respective first monitoring device 140a and second monitoring device 140b (generally, monitoring devices 140). As will be appreciated, other exercises using different equipment with various biometric and monitoring devices are contemplated by the present disclosure.

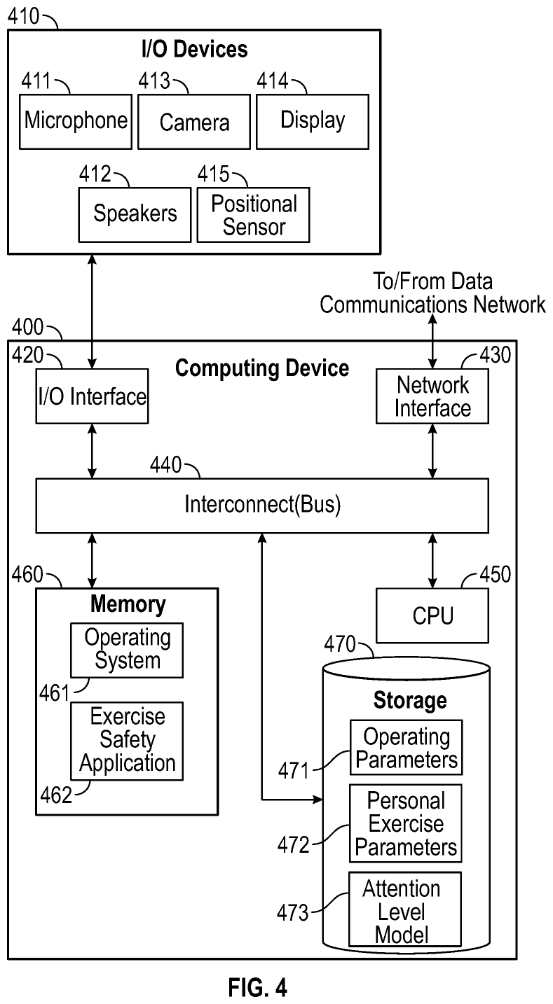

In various embodiments, the monitoring device 140 may be integrated into the ED 120 as a component of the ED 120, or may be a separate device, such as a smartphone or tablet associated with the user 110 and connected (temporarily) with the ED 120. In various embodiments, the monitoring device 140 may be placed in communication with the ED 120 and/or the biometric device 130 by one or more wireline or wireless communications channels (e.g., a Universal Serial Bus (USB) connection, a BLUETOOTH® connection). Example computing hardware, as may be used in the ED 120, biometric device 130, and/or monitoring device 140 is discussed in greater detail in regard to FIG. 4.

Each monitoring device 140 monitors how the user 110 is using the associated ED 120 using various sensors, such as cameras, microphones, gyroscopes, and accelerometers as well as data provided by the ED 120 or the biometric device 130. For example, a monitoring device 140 can use a camera to determine a facial expression, pattern of exercise, direction of focus, and the like, and use a microphone to determine a pattern of breathing/speech, pattern of exercise, the presence of distractions (e.g., a television or another person speaking nearby), and the like. The monitoring device 140 is in communication with the ED 120, and can receive system settings for the ED 120 (e.g., incline, speed, resistance/weight, upcoming adjustments to settings), receive usage data from the ED 120, and send commands to adjust system setting to the ED 120. The biometric device 130, which may include a smart watch, a pacemaker, an insulin pump, a smartphone, a pedometer, or the like, provides the monitoring device 140 with biometric data related to the user 110 including, for example, heart rate, blood pressure, skin conductivity (e.g., via a galvanic response), blood sugar, etc.

The monitoring device 140 may monitor the body position of a user 110 relative to an ED 120, a range of motion of the user 110 when interacting with the ED 120, and vital signs of the user 110 to identify the physical state of the user 110. The monitored vital signs may include a breathing pattern (measured by a microphone or gyroscope), a speed of a limb engaged in a repetitive motion (measured by image recognition or an accelerometer), a pain level (measured by facial recognition), a skin temperature (measured by a thermometer), sweat production rate (measured by a galvanic sensor or image recognition), and heart rate (measured by a heart rate monitor), among other physical characteristics.

The monitoring device 140 may monitor the direction of gaze (e.g., where the user 110 is looking), facial expression, and vocalizations from the user 110 (e.g., labored breathing, conversational pattern) to identify the attentiveness level of the user 110. In various embodiments, the physical state of the user 110 is also used to determine the attentiveness level of the user 110. For example, the first user 110a is shown with an upright body position, whereas the second user 110b is shown with a slouched body position, which may indicate that the second user 110b has a fatigued attentiveness level compared to the first user 110a who has a more alert attentiveness level.

In some embodiments, the ED 120 reports settings selected by the user 110 to perform the exercise or a selected exercise regime. The manner in which the user 110 is using the ED 120 includes the physical information of the actions of the user 110, specific to that ED 120 as well as operational data for the state of that ED 120. For example, for a treadmill, the monitoring device 140 tracks the manner in which the user 110 runs (e.g., the running gait, path of locomotion, foot angles at footfall, arm swing, posture, etc.), and the ED 120 reports the speed and incline for the track of the treadmill. In another example, for a lateral arm raise machine, the monitoring device 40 tracks the manner in which the user's arms raise (e.g., how evenly the left and right arms raise, how far the arms raise, how long a repetition from lowered-to-raised-to-lowered lasts, how evenly each repetition moves the arm bars, whether the user 110 is slamming the arm bars at the top or bottom of a repetition), and the ED 120 reports the weight/resistance and seat height/angle of the lateral arm raise machine.

The monitoring device 140 determines, based on the physical data related to the user 110 and the operational data related to the ED 120, the physical state and attentiveness level of the user 110 to determine how the user 110 is feeling while performing the exercise, and whether to intervene by adjusting the exercise to a safer or more appropriate level for the given user 110. In some embodiments, the monitoring device 140 monitors the user's mental and physical states to determine a general medical condition of the user 110, while in other embodiments, the user 110 may indicate a particular medical condition that the monitoring device 140 is to monitor. For example, a user 110 who has a pacemaker may indicate that the monitoring device 140 is to observe breathing rate and skin flush to ensure that the user 110 is exercising within the capabilities of the pacemaker. In another example, a user 110 who has a history of ankle sprains may indicate that the monitoring device 140 is to focus on the running gait, footfalls, and speed of the user 110 when exercising on a treadmill 110.

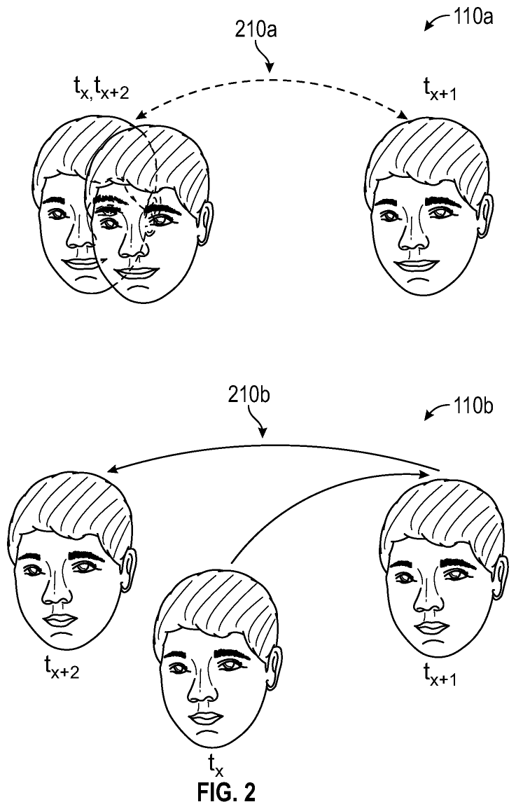

FIG. 2 illustrates a time lapse view of the range of motion for the first user 110a and the second user 110b. Travels paths 210a-b (generally, travel path 210) from time tₓ to time tₓ₊₂ for the head of the first user 110a and for the second user 110b respectively are illustrated in FIG. 2, but embodiments tracking the legs, arms, feet, shoulders, or other body parts are contemplated. By tracking the motion of a body part for a set period of time or number of repetitions, the monitoring device 140 can determine how consistently the user 110 is moving and a range of motion for the user 110, which may be used to infer the attentiveness level of that user 110.

For example, the first travel path 210a for the first user 110a shows the user's head moving in a back-and-forth and up-and-down motion, as is typical when running. The first travel path 210a exhibits less variance than the second travel path 210b for the second user 110b. The monitoring devices 140, having collected the travel paths 210, may thus determine that the second user 110b is struggling to perform on the second ED 120b, due to being tired, distracted, or having selected a setting on the second ED 120b beyond the capabilities of the second user 110b. Accordingly, to mitigate the risk of injury for the second user 110b, the second monitoring device 140b may adjust one or more settings on the second ED 120b to choose a less strenuous exercise for the second user 110b than is currently selected.

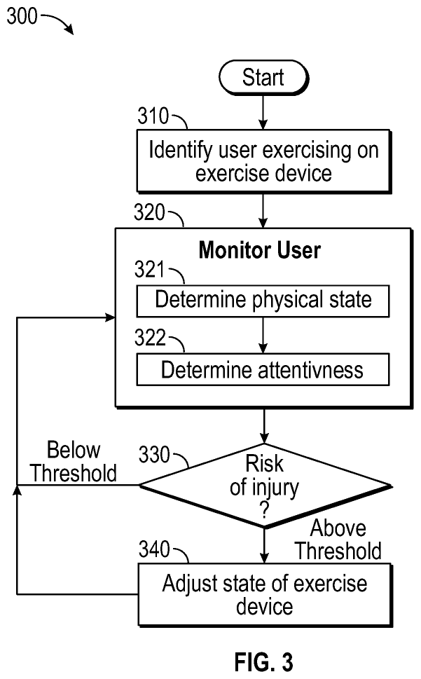

FIG. 3 is a flowchart of a method 300 for applying attention and exertion based safety mechanisms in exercise devices. According to method 300, the monitoring device 140 gathers information related to a user 110 exercising on an ED 120 and adjusts the settings of the ED 120 from a first state to a second state when a risk of injury to a user 110 exceeds a predefined threshold, based on the physical and/or mental state of the user 110.

Method 300 begins with block 310, where the monitoring device 140 identifies a user 110 exercising on an ED 120, and a state of the ED 120 at which the user 110 is using the ED 120. For example, a state of an ED 120 of a rowing machine may include a seat position, a line resistance, and an angle of seat travel. In another example, an ED 120 of a treadmill may include a track incline and speed. In various embodiments, the user 110 may be identified as a specific individual person, for whom the monitoring device 140 maintains an individual profile (e.g., including a medical record, exercise profile, facial/body profile) or as a general person (e.g., as “runner123” versus “John Doe”).

At block 320, the monitoring device 140 monitors a physical state and an attentiveness level of the user 110 while the ED 120 is in the initial state. At sub-block 321, the monitoring device 140 determines the physical state of the user 110, and at sub-block 322, the monitoring device 140 determines the attentiveness level of the user 110.

At sub-block 321, the monitoring device 140 directly observes some aspects of the physical state of the user 110 via cameras and/or microphones to determine the physical state of the user 110, and may also receive data from a biometric device 130 and/or the ED 120 related to the physical state of the user 110 to refine or provide additional data on the physical state of the user 110. The monitoring device 140 may monitor one or more of a body position of the user 110 relative to the ED 120, a range of motion of the user 110 interacting with the ED 120 over a period of time, and vital signs of the user 110. In various embodiments, the monitoring device 140 may determine or measure the vital signs directly or collect vital signs from a biometric device 130 or the ED 120, such as, but not limited to: a breathing pattern; a speed of repetitive motion in a limb; a skin temperature; a sweat production rate; and a heart rate.

At sub-block 322, the monitoring device 140 determines the attentiveness level of the user 110. In various embodiments, the monitoring device 140 uses a machine learning model that correlates various physical states or physical data to various attentiveness levels. For example, a facial expression may be correlated with a level of pain experienced by the user 110, or a breathing rate may be correlated to a level of fatigue experienced by the user 110. The monitoring device 140 may determine the attentiveness level of the user 110 based on one or more of: a direction of gaze and where the user 110 is looking; breathing patterns (e.g., even vs. ragged, deep vs. shallow, loud vs. quiet, nasal vs. oral); speech patterns and how the user is speaking (e.g., loud vs. quiet, halting vs. steady, generally vocal vs. normally silent); speech content (e.g., “oww,” “ouch,” counting, full sentences); and facial expressions. The machine learning model of the attentiveness level of the user 110 may be individualized for a particular person (e.g., Jane Doe) or may be generalized for a given demographic group (e.g., persons of a given weight, height, age, gender, etc.) to determine the attentiveness level of the user 110. For example, a first person may exhibit a given facial expression when in pain that is similar to a facial expression that a second person exhibits when concentrating, and different individualized machine learning models for the first and second persons can differentiate the facial expressions to determine the correct attentiveness levels for the associated persons that may otherwise be misconstrued.

When monitoring the gaze of the user 110, the monitoring device 140 may monitor one or more of the consistency and the target of the gaze. The target of the gaze may include the ED 120 (or a specific portion thereof, such as a output screen of the ED 120, a track of a treadmill, etc.) or an object in the environment other than the ED 120, such as, for example, a television, a mirror (e.g., for the user 110 to monitor form in performing the exercise), a biometric device 130, a clock, a trainer, a spotter, or other person, etc.

Depending on the exercise or portion thereof, the monitoring device 140 may determine that the user 110 is focused or unfocused. For example, a user 110 of an ED 120 of a rowing machine whose gaze is targeted to a television off center from the rowing track may not be focused on the rowing motion and be more prone to injury (e.g., due to poor form) than a user 100 of whose gaze is targeted directly ahead. In another example, a user 110 who is adjusting the ED 120 (e.g., increasing or decreasing speed, incline, resistance, weight, etc.) may be determined to have an unfocused attentiveness level when the gaze is not directed to a control interface, such as when a user 110 is talking to a friend instead of paying attention to how the ED 120 is being adjusted. Accordingly, the monitoring device 140 monitors the targets of the gaze of the user 110 over a rolling time window to determine when the user 110 is attentive or inattentive to the exercise and the ED 120 and thus when the user 100 is at a lower or higher risk of injury.

Because a mentally focused user 110 is less likely to be injured when using an ED 120 than a mentally unfocused user 110, the monitoring device 140 also tracks the consistency of the gaze of the user 110 when determining the attentiveness level of the user 110. Accordingly, when tracking the gaze of a user 110, the gaze consistency measures how long a gaze of the user 110 rests on a given target in the environment. For example, a first user 110a who has a gaze directed to a television for at least X % of the time in a given time window while using a first ED 120, with quick glances to a watch, a friend on a second ED 120b, etc. may be determined to have a consistent gaze when Xis greater than a predefined attention threshold. In another example, a second user who has a gaze directed to the track of a treadmill, a mirror in front of the treadmill, a friend nearby etc., with no one target exceeding a predefined attention threshold within a time window, may be determined to have an inconsistent gaze and to therefore have an unfocused attentiveness level. The monitoring device 140 may set different lengths of for the attention threshold based on the ED 120 or a setting of the ED 120. For example, a treadmill may have a longer attention threshold when set to a higher speed than when set to a lower speed, so that the user 110 is required to provide more consistent gaze focus within a time window to maintain a higher speed on the treadmill.

At block 330, the monitoring device 140 determines, based on the physical state and attentiveness level of the user 110, whether the user 110 is experiencing a risk of injury above a predefined threshold while the ED 120 is operating in the first state. The monitoring device 140 compares the vital signs and attentiveness levels of the user 110 against a demographic thresholds to determine whether the vital signs or mental indicate a generalized increase to the risk of injury for persons matching the demographic profile and compares the vital signs and determined attentiveness level of the user 110 against the personalized thresholds to determine whether the vital signs or attentiveness level indicate a specific increase to the risk of injury for the user. The monitoring device 140 may determine the thresholds based on device-specific thresholds for the ED 120 and personalized thresholds for the user 110. The device-specific thresholds may specify a location of the user 110 relative to the ED 120 for safe usage (e.g., a center of gravity to avoid tipping the ED 120, keeping appendages of the user 110 out of pinch points of mechanisms of the ED 120, identifying improper usage of the ED 120), a range of motion that is considered safe on the ED 120, or the like. The personalized thresholds may be set for a demographic group (e.g., persons with a Body Mass Index with a given range, persons of a given height range, persons of a given age, etc.) or a particular individual. In some embodiments, the personalized thresholds are identified from a medical history associated with the user 110 that specifies a medical condition with an associated personalized threshold that is different than or in addition to those personalized thresholds set for a demographic group or generalized person.

In response to a negative determination at block 330 (i.e., that the risk of injury for the user 110 is below the threshold), method 300 may return to block 320 to continue monitoring the user 110.

In response to a positive determination at block 330 (i.e., that the risk of injury for the user is above the threshold), method 300 proceeds to block 340, where the monitoring device 140 adjusts the ED 120 from the first state to a second state to alleviate the risk of injury. In some embodiments, the second state is a less-intensive form of an exercise performed when the ED 120 is in the first state (e.g., lower weight/resistance, slower speed, lower incline), while in other embodiments the second state is a stopped state (e.g., no speed, locking the device to hold weight off of the user 110). As will be appreciated, the transition from the first state to the second state may be gradual and/or preceded by an alert to the user 110 to help avoid injury to the user 110 due to a sudden or unexpected shift in operations of the ED 120. When the second state allows for continued exercise operations, method 300 may return to block 320 to continue monitoring the user 110 while using the ED 120 at the second (or subsequent) state. When the second state stopes the exercise, method 300 may conclude or return to block 320 to continue monitoring the user 110 until the physical state or attentiveness level that led to the stopped state is resolved, and monitored exercise may then resume.

FIG. 4 illustrates a computing system 400, which may be: a personal computer; a laptop; a tablet; a smartphone; a computing device integrated in an ED 120, biometric device 130, or monitoring device 140; etc. As shown, the computing system 400 includes, without limitation, a central processing unit (CPU) 450, a network interface 430, an interconnect 440, a memory 460, and storage 470. The computing system 400 may also include an I/O device interface 420 connecting I/O devices 410 to the computing system 400. The I/O devices 410 include, but are not limited to, a microphone 411 for audio input, speakers 412 for audio output, a camera 413 for visual input, a display 414 for visual output, contact sensors 415 for measuring the body conditions of a person holding the contact sensors 415 (e.g., skin temperature, galvanic skin response measurements), and positional sensors 416 for spatial input (e.g., accelerometers, gyroscopes, magnetometers, Global Positioning System receivers).

The CPU 450 retrieves and executes programming instructions stored in the memory 460. Similarly, the CPU 450 stores and retrieves application data residing in the memory 460. The interconnect 440 facilitates transmission, such as of programming instructions and application data, between the CPU 450, I/O device interface 420, storage 470, network interface 440, and memory 460. CPU 450 is included to be representative of a single CPU, multiple CPUs, a single CPU having multiple processing cores, and the like. And the memory 460 is generally included to be representative of a random access memory. The storage 470 may be a disk drive storage device. Although shown as a single unit, the storage 470 may be a combination of fixed and/or removable storage devices, such as magnetic disk drives, flash drives, removable memory cards or optical storage, network attached storage (NAS), or a storage area-network (SAN). The storage 470 may include both local storage devices and remote storage devices accessible via the network interface 430.

Further, computing system 400 is included to be representative of a physical computing system as well as virtual machine instances hosted on a set of underlying physical computing systems. Further still, although shown as a single computing system, one of ordinary skill in the art will recognized that the components of the computing system 400 shown in FIG. 4 may be distributed across multiple computing systems connected by a data communications network. For example, the computing system 400 may represent components of the ED 120, the biometric device 130, or monitoring device 140 linked via a BLUETOOTH® or other communications protocol via associated network interfaces 430.

As shown, the memory 460 includes an operating system 461 (e.g., Microsoft's WINDOWS® Operating System) and an exercise safety application 462. The exercise safety application 462 includes programmatic instructions to perform the functionalities of the monitoring device 140 described herein. The exercise safety application 462 accesses one or more operating parameters 471 for how to safely use a given ED 120 that specify one or more device-specific safety thresholds (e.g., ranges of motion, speeds of operation, decibel levels of usage).

The exercise safety application 462, in some embodiments, accesses a personal exercise parameter 472 for a given user 110 (e.g., a medical record, a personal fitness goal, biographic data (age, height, weight, arm span, waist size, etc.) to determine personalized safety thresholds for the given user 110. The exercise safety application 462 gathers physical state data related to the user 110 while exercising on the ED 120 to determine when an aspects of the physical state of the user 110 exceeds a (device-specific or personalized) safety threshold. For example, when the user 110 is positioned off a center of gravity for using the ED 120, the exercise safety application 462 may determine that the user 110 has exceeded a device-specific safety threshold related to tipping the ED 120 over. In another example, when a first user 110 and a second user 110 both have heart rates X beats per minute, the exercise safety application 462 may determine that the first user 110 has exceeded a personalized safety threshold and the second user 110 has not exceeded a personalized safety threshold based on the personal data for the individual users 110.

The exercise safety application 462 provides an attention level model 473 with the physical state data gathered about the user 110 while exercising on the ED 120 to determine an attentiveness level of the user 110. For example, attention level model 473 receives data related to the user 110 (e.g., facial expression, body language, gaze, vocal patterns, motion patterns) and returns an attentiveness level for the administering individual 110. The exercise safety application 462 compares the determined attentiveness level against the safety thresholds to determine when the attentiveness level of the user 110 may elevate risk of injury on the ED 120. For example, when a facial expression of the user 110 is correlated to the user 110 being in pain, the exercise safety application 462 may determine that the user 110 has exceeded a personalized safety threshold. In another example, when a gaze of the user 110 is directed away from the ED 120 and is correlated to the user 110 being distracted, the exercise safety application 462 may determine that the user 110 has exceeded a device-specific safety threshold for the safe operation of the ED 120.

When the exercise safety application 462 determines that one or more of the physical state and the attentiveness level of the user 110 exceeds a safety threshold, the exercise safety application 462 transmits a control signal of the ED 120 to engage a safety mechanism based on the mental and/or physical state of the user 110. Because a sudden stop, locking of the ED 120, or other change in state may lead to injury, (e.g., a distracted runner may be unprepared for a treadmill to stop, and trip), the control signal may gradually adjust the ED 120 from a first state to a second state (which may include an eventual stop) that is safer for the user 110. For example, when a runner on a treadmill set to X speed at Y angle of incline in a first state is identified as being at elevated risk of injury, the exercise safety application 462 may gradually adjust the treadmill over Z seconds to a second state of X/2 speed at Y/2 angle of incline. If the runner continues to exhibit a mental or physical state associated with an increased risk of injury, the exercise safety application 462 may then adjust the treadmill from the second state to a stopped state of 0 speed at 0 angle of incline over another period of time.

Thus the exercise safety application 462 provides improve the safety of exercise equipment by monitoring the physical state and attentiveness level of the persons using that equipment to automatically adjust operations of the exercise equipment.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the following, reference is made to embodiments presented in this disclosure. However, the scope of the present disclosure is not limited to specific described embodiments. Instead, any combination of the following features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.”

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing is directed to embodiments of the present invention, other and further embodiments of the invention may be devised without departing from the basic scope thereof, and the scope thereof is determined by the claims that follow.

What is claimed is:

1. A method, comprising:
identifying a user exercising on an exercise device while the exercise device is in a first state;
monitoring a physical state and an attentiveness level of the user while the exercise device is in the first state, wherein monitoring the attentiveness level includes monitoring targets of a gaze of the user within a time window and a consistency of the gaze within the time window;
determining, based on the physical state and the attentiveness level of the user, that the user is experiencing a risk of injury above a predefined threshold; and
adjusting the exercise device from the first state to a second state to alleviate the risk of injury.

2. The method of claim 1, wherein monitoring the physical state includes:
monitoring a body position of the user relative to the exercise device;
monitoring a range of motion of the user interacting with the exercise device over a period of time; and
monitoring vital signs of the user.

3. The method of claim 2, wherein the vital signs include:
a breathing pattern;
a speed of repetitive motion in a limb;
a pain level;
a skin temperature;
a sweat production rate; and
a heart rate.

4. The method of claim 3, further comprising:
identifying, from a medical history associated with the user, a medical condition;
wherein monitoring the physical states includes:
comparing the vital signs against the medical history for the user to determine whether the vital signs indicate a specific increase to the risk of injury for the user; or
comparing the vital signs against a demographic profile for the user to determine whether the vital signs indicate a generalized increase to the risk of injury for persons matching the demographic profile.

5. The method of claim 1, wherein monitoring the attentiveness level of the user includes:
monitoring breathing patterns of the user;
monitoring speech patterns of the user;
monitoring speech content of the user; and
monitoring a facial expression of the user.

6. The method of claim 1, wherein adjusting the exercise device from the first state to the second state selects a less-intensive form of an exercise performed when the exercise device is in the first state.

7. The method of claim 6, further comprising:
monitoring the physical state and the attentiveness level of the user while the exercise device is in the second state;
determining, based on the physical state and the attentiveness level of the user, that the user continues to experience the risk of injury above the predefined threshold while the exercise device is in the second state; and
adjusting the exercise device from the second state to a stopped state based on one of the physical state and the attentiveness level.

8. A system, comprising:
a processor; and
a memory including computer-readable program code that when executed by the processor causes the system to perform an operation comprising:
identifying a user exercising on an exercise device while the exercise device is in a first state;
monitoring a physical state and an attentiveness level of the user while the exercise device is in the first state, wherein monitoring the attentiveness level includes monitoring targets of a gaze of the user within a time window and a consistency of the gaze within the time window;
determining, based on the physical state and the attentiveness level of the user, that the user is experiencing a risk of injury above a predefined threshold; and
adjusting the exercise device from the first state to a second state to alleviate the risk of injury.

9. The system of claim 8, wherein monitoring the physical state includes:
monitoring a body position of the user relative to the exercise device;
monitoring a range of motion of the user interacting with the exercise device over a period of time; and
monitoring vital signs of the user.

10. The system of claim 9, wherein the vital signs include:
a breathing pattern;
a speed of repetitive motion in a limb;
a pain level;
a skin temperature;
a sweat production rate; and
a heart rate.

11. The system of claim 10, further comprising:
identifying, from a medical history associated with the user, a medical condition;
wherein monitoring the physical states includes:
comparing the vital signs against the medical history for the user to determine whether the vital signs indicate a specific increase to the risk of injury for the user; or
comparing the vital signs against a demographic profile for the user to determine whether the vital signs indicate a generalized increase to the risk of injury for persons matching the demographic profile.

12. The system of claim 8, wherein monitoring the attentiveness level of the user includes:
monitoring breathing patterns of the user;
monitoring speech patterns of the user;
monitoring speech content of the user; and
monitoring a facial expression of the user.

13. The system of claim 8, wherein adjusting the exercise device from the first state to the second state selects a less-intensive form of an exercise performed when the exercise device is in the first state.

14. The system of claim 13, further comprising:
monitoring the physical state and the attentiveness level of the user while the exercise device is in the second state;
determining, based on the physical state and the attentiveness level of the user, that the user continues to experience the risk of injury above the predefined threshold while the exercise device is in the second state; and
adjusting the exercise device from the second state to a stopped state based on one of the physical state and the attentiveness level.

15. A computer program product comprising a computer-readable storage medium having computer-readable program code embodied therewith, the computer-readable program code executable by one or more computer processors to:
identify a user exercising on an exercise device while the exercise device is in a first state;
monitor a physical state and an attentiveness level of the user while the exercise device is in the first state, wherein monitoring the attentiveness level includes monitoring targets of a gaze of the user within a time window and a consistency of the gaze within the time window;
determine, based on the physical state and the attentiveness level of the user, that the user is experiencing a risk of injury above a predefined threshold; and
adjust the exercise device from the first state to a second state to alleviate the risk of injury.

16. The computer program product of claim 15, wherein to monitor the physical state the computer-readable program code is further executable by the one or more computer processors to:
monitor a body position of the user relative to the exercise device;
monitor a range of motion of the user interacting with the exercise device over a period of time; and
monitor vital signs of the user.

17. The computer program product of claim 16, wherein the vital signs include:
a breathing pattern;
a speed of repetitive motion in a limb;
a pain level;
a skin temperature;
a sweat production rate; and
a heart rate.

18. The computer program product of claim 17, wherein the computer-readable program code is further executable by the one or more computer processors to:
identify, from a medical history associated with the user, a medical condition;
wherein to monitor the physical states the computer-readable program code is further executable by the one or more computer processors to:
compare the vital signs against the medical history for the user to determine whether the vital signs indicate a specific increase to the risk of injury for the user; or
compare the vital signs against a demographic profile for the user to determine whether the vital signs indicate a generalized increase to the risk of injury for persons matching the demographic profile.

19. The computer program product of claim 15, wherein to monitor the attentiveness level of the user, the computer-readable program code is further executable by the one or more computer processors to:
monitor breathing patterns of the user;
monitor speech patterns of the user;
monitor speech content of the user; and
monitor a facial expression of the user.

20. The computer program product of claim 15, wherein adjusting the exercise device from the first state to the second state selects a less-intensive form of an exercise performed when the exercise device is in the first state.
